(19) United States
(12) Patent Application Publication
(10) Pub. No.: US 2008/0287481 A1
(43) Pub. Date: Nov. 20, 2008

(54) PROCESS FOR THE PREPARATION OF DESLORATADINE POLYMORPH MIXTURES

(76) Inventors: Mayur Devjibhai Khunt, Junagadh (IN); Srinivasa Reddy Madduri, Guntur (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/851,970

(22) Filed: Sep. 7, 2007

Related U.S. Application Data
(60) Provisional application No. 60/870,002, filed on Dec. 14, 2006.

(30) Foreign Application Priority Data
Sep. 8, 2006 (IN) 1638/CHE/2006

(51) Int. Cl. A61K 31/435; C07D 221/06; A61P 43/00

(52) U.S. Cl. 514/290; 546/93

(57) ABSTRACT

The present application provides a process for the preparation of mixture of polymorphic Form I and Form II of desloratadine in any desired ratio.

### FIELD OF THE APPLICATION

The present application relates to a process for the preparation of mixture of polymorphic Form I and Form II of desloratadine and compositions containing them.

### BACKGROUND OF THE APPLICATION

Desloratadine is chemically known as 8-chloro-6,11-dihydro-11-(4-piperidylidene)-5H-benzo[5,6]cyclohepta[1,2-b]pyridine (herein after referred by its adopted name desloratadine) and is represented by the structural Formula I.

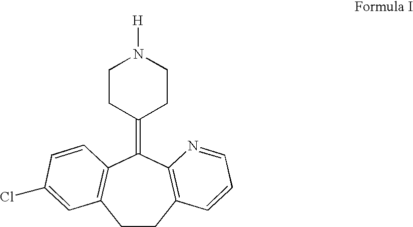

Desloratadine is a metabolic derivative of loratadine. Desloratadine is available commercially in the United States by the brand name CLARINEX as film coated tablets containing 5 mg desloratadine.

It is advantageous to have a single process which can give any desired ratio of the two polymorphic forms I and II by varying the process parameters.

### SUMMARY OF THE APPLICATION

An embodiment of the present invention provides a process which gives any desired ratio of the mixture of polymorphic Form I and Form II of desloratadine by controlling the process parameters.

In one embodiment, the present invention relates to a process for the preparation of an intimate mixture of desloratadine polymorphs Form I and Form II.

In another embodiment, the present invention relates to a process for the preparation of an intimate mixture of desloratadine polymorphs Form I and Form II in any desired ratio of either Form to the other.

In an embodiment, the process for the preparation of a mixture of desloratadine polymorphic Form I to Form II, in a ratio of either form to the other, comprises:

a) providing a solution of desloratadine in a hydrocarbon solvent;

b) concentrating the solution obtained in step a) to a specific volume; and

c) isolating the desired mixture of forms.

In an embodiment, the process of the present invention is industrially scaleable, and economic.

In an embodiment the present invention provides mixtures of different polymorphic forms in specified ratios can be used in the preparation of pharmaceutical formulations

In another embodiment, the present invention provides a pharmaceutical composition comprising the mixture of desloratadine crystalline Form I and Form II prepared by the process of the present invention, having a desired weight ratio of Form I to Form II and at least one pharmaceutical excipient.

### BRIEF DESCRIPTION OF THE DRAWINGS

FIG. 1 is an XRPD pattern for pure desloratadine polymorphic Form I.

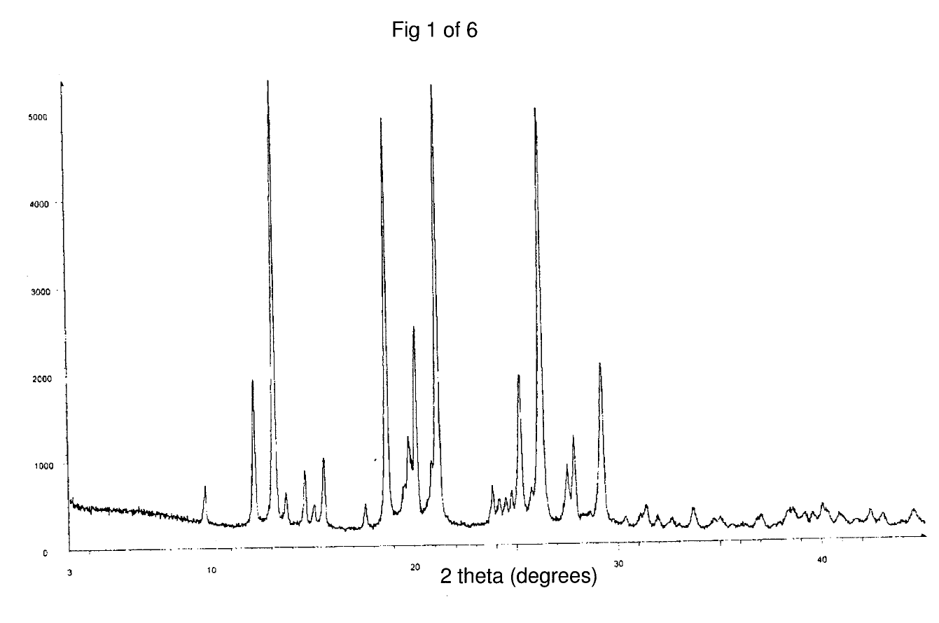

FIG. 2 is an XRPD pattern for pure desloratadine polymorphic Form II.

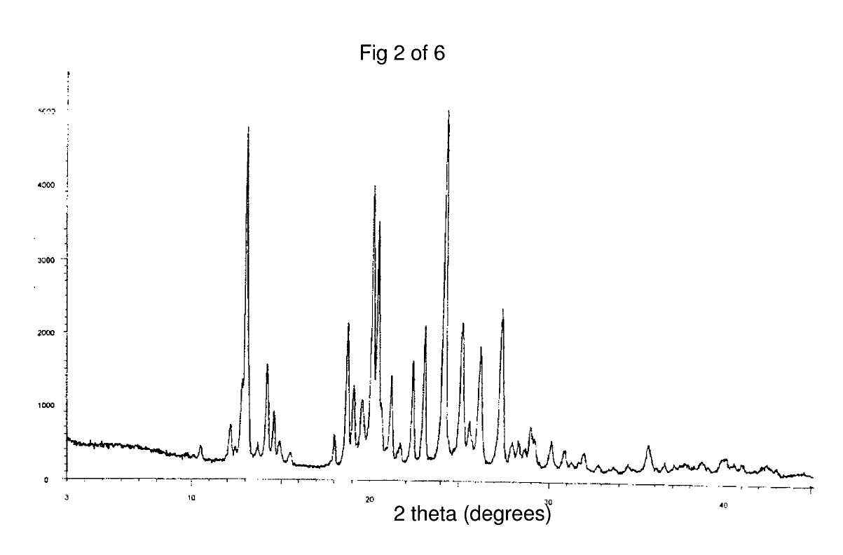

FIG. 3 is an XRPD pattern of a mixture of desloratadine polymorphic Forms I and II in a ratio of about 80:20 w/w.

FIG. 4 is an XRPD pattern of a mixture of desloratadine polymorphic Forms I and II in a ratio of about 70:30 w/w.

FIG. 5 is an XRPD pattern of a mixture of desloratadine polymorphic Forms I and II in a ratio of about 60:40 w/w.

FIG. 6 is a graph showing variation in the percentage of Form II in product by varying the percentage of the solvent distilled from a solution.

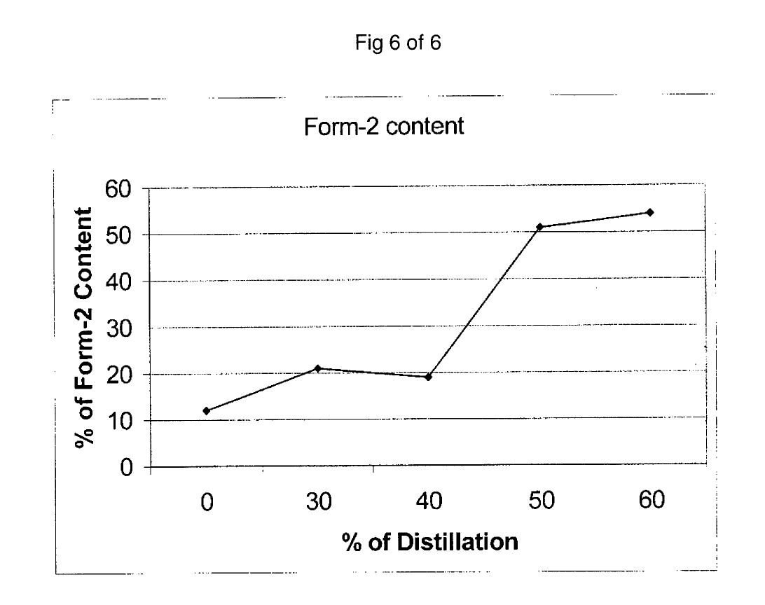

### DETAILED DESCRIPTION OF THE APPLICATION

In an embodiment, the present invention relates to a process for the preparation of an intimate mixture of desloratadine polymorphs Form I and Form II.

In one embodiment, the present invention relates to a process for the preparation of an intimate mixture of desloratadine polymorphs Form I and Form II in a ratio of either form to the other as desired, characterized by the X-ray powder diffraction (XRPD) pattern.

The term “intimate mixture,” as used herein, unless otherwise indicated, means that the mixture of desloratadine polymorphs Form I and Form II in any desired ratio has an evenly dispersed, uniform and stable mixture of the polymorphs and has the same composition of the polymorphs throughout the mixture.

The term “any desired ratio” as used herein, unless otherwise indicated, includes variable ratios of Form I to Form II. In an embodiment of the present invention the ratio of Form I to Form II is between about 15:85 and 85:15 by weight. In an embodiment of the present invention the ratio of Form I to Form II is between about 20:80 and 80:20 by weight. In an embodiment of the present invention the ratio of Form I to Form II is between about 30:70 and 70:30, by weight.

The amounts of Form I and Form II of desloratadine are expressed throughout the application as weight ratios or as weight percent. Weight percent is intended to mean: [(either Form I or Form II weight)÷(Form I+Form II weight)]×100.

The above mentioned mixtures of crystalline forms of desloratadine are characterized by their X-ray powder diffraction (“XRPD”) patterns. All XRPD information herein was generated on a Bruker AXS, D8 Advance Powder X-ray Diffractometer with a Cu K alpha-1 radiation source.

The XRPD patterns of the mixtures of forms have the characteristic peaks of both crystalline Form I and Form II of desloratadine. Depending on the weight ratios of the forms, the intensities of characteristic peaks will vary.

In an embodiment of the present invention, the crystalline Form I of desloratadine is characterized by its XRPD pattern having significant peaks at about 12.1, 13.1, 18.6, 20.0, 20.7, 21.1, and 25.1, ±0.2 degrees 2θ. It is also characterized by additional XRPD peaks at about 26.2, and, 29.2, ±0.2 degrees 2θ.

In an embodiment of the present invention, the crystalline Form II of desloratadine is characterized by its XRPD pattern having significant peaks at about 12.7, 13.0, 14.1, 18.6, 20.0, 20.3, 21.6, 23.0, 24.1, and 25.1, ±0.2 degrees 2θ. It is also characterized by additional XRPD peaks at about 26.1, and 27.3, ±0.2 degrees 2θ.

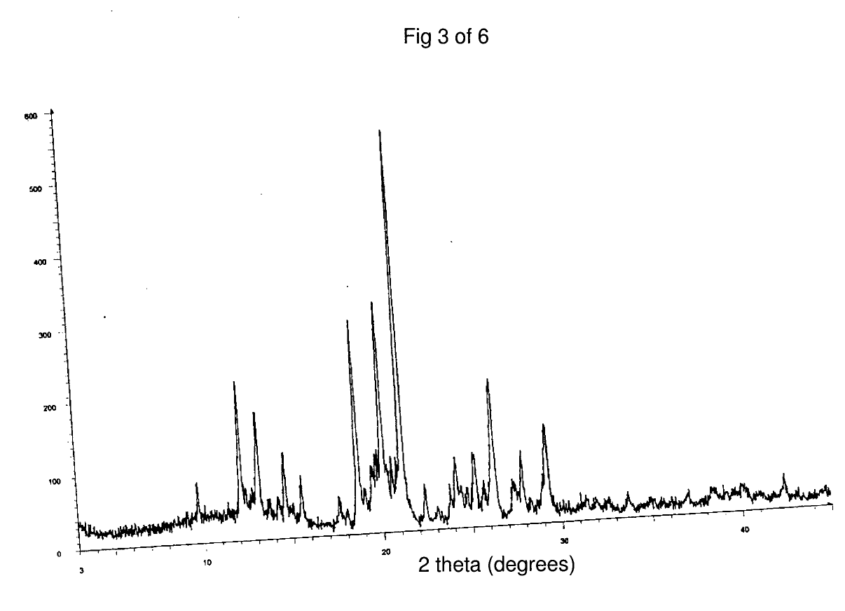

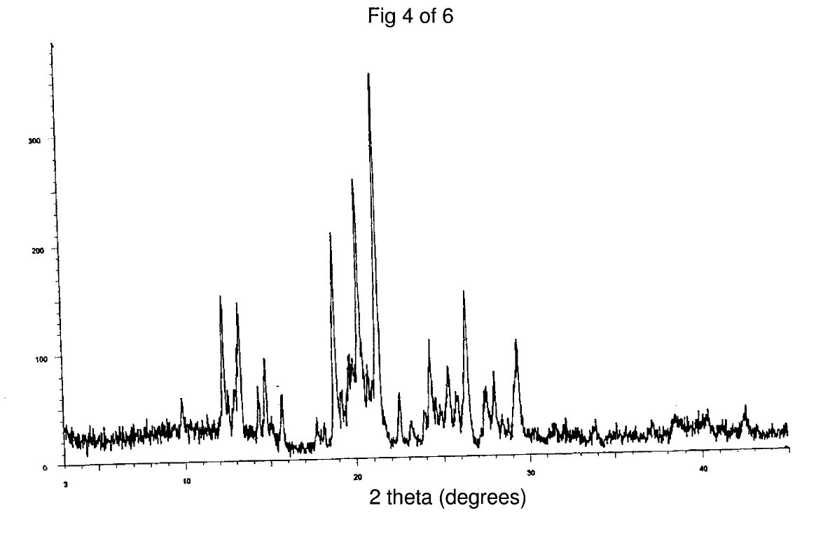

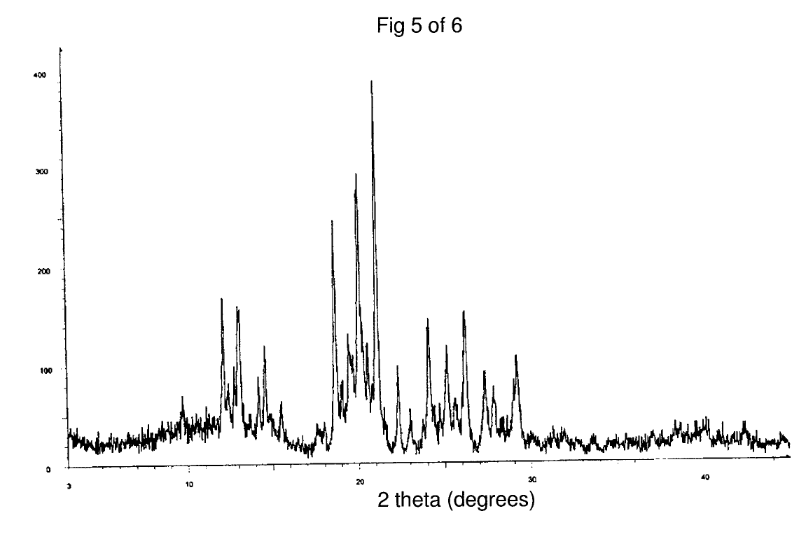

Characteristic XRPD patterns for mixtures having ratios of Form I to Form II of about 80% w/w to about 20% w/w is shown in FIG. 3; about 70% to about 30% w/w as shown in FIG. 4 and about 60% to about 40% w/w as shown in FIG. 5.

In embodiment of the present invention, the process for the preparation of an intimate mixture of desloratadine polymorphic Form I to Form II, in a ratio of either form to the other as desired, comprises:

a) providing a solution of desloratadine in a hydrocarbon solvent;

b) concentrating the solution obtained in step a) to a specific volume; and

c) isolating the desired mixture of forms.

Step (a)—Providing a Solution of Desloratadine.

The solution of desloratadine may be obtained by dissolving desloratadine in a suitable solvent, or such a solution may be obtained directly from a reaction in which desloratadine is formed. Desloratadine may be prepared by any of the methods described in U.S. Pat. No. 4,659,716, U.S. Pat. No. 4,826,853 and U.S. Pat. No. 5,925,648 which describe desloratadine or its pharmaceutically acceptable salts and its pharmaceutical composition; U.S. Pat. No. 6,506,767 which discloses substantially pure crystalline forms of desloratadine designated as Form I and Form II, and processes for their preparation; and U.S. Patent Application Publication No. 2006/0223841 which discloses processes for the preparation of crystalline Form I and mixtures of various ratios of crystalline Form I and Form II of desloratadine.

When the solution is prepared by dissolving desloratadine in a suitable solvent, any form of desloratadine such as a crystalline or amorphous form, including any salts, solvates and hydrates may be utilized for preparing the solution.

Suitable hydrocarbon solvents that can be used for the dissolution of desloratadine include, but are not limited to butane, pentane, hexane, isopentane, cyclohexane, toluene, xylene, n-heptane, n-hexane, and the like or mixtures thereof. In an embodiment of the present invention, the solvent is cyclohexane.

The dissolution temperatures can range from about 20 to about 120° C. depending on the solvent used for dissolution. Any other temperature is also acceptable as long as a clear solution of desloratadine is provided.

The quantity of solvent used for dissolution can range from about 20 times to about 50 times to the weight of desloratadine taken. The concentration of desloratadine in the solution may generally range from about 0.5 to about 5 g/ml in the solvent. Optionally, the solution obtained above can be filtered to remove the undissolved particles before proceeding for further processing.

The undissolved particles can be removed suitably by filtration, centrifugation, decantation, and other techniques. The solution can be filtered by passing through paper, glass fiber, or other membrane material, or a bed of a clarifying agent such as celite. Depending upon the equipment used and the concentration and temperature of the solution, the filtration apparatus may need to be preheated to avoid premature crystallization.

### Step (B)—Concentrating the Solution Obtained in Step (a).

The solution may be concentrated suitably using techniques such as evaporation, atmospheric distillation, or distillation under vacuum. Any temperature ranging from about 20° C. to about 120° C. can be used for concentration of the solvent. In an embodiment of the present invention the concentration is carried out by distillation.

The quantity of solvent distilled, and the ultimate concentration of desloratadine remaining in the solution determines the percentages of the polymorphic Forms I and II in the final product.

When the concentration of desloratadine in the solution gradually drops from about 0.5 g/ml to about 0.3 g/ml or below, the percentage of Form II in the product increases, and when the concentration is increased from about 0.5 g/ml to about 0.9 g/ml, the percentage of Form I in the product gradually increases. Other parameters like the duration of distillation and the temperature of distillation may also play a role in determining the ratio of Form II in Form I in the product.

In an embodiment of the present invention, when the concentration of the solution is about 0.1 g/ml, to about 0.15 g/ml, the ratio of Form I to Form II obtained in the product is about 70:30. In an embodiment of the present invention when the concentration of the solution is from about 0.05 g/ml to about 0.1 g/ml, the ratio of Form I to Form II in the obtained product is about 60:40. In another embodiment of the present invention when the concentration of the solution is from about 0.05 g/ml to about 0.01 g/ml, the ratio of Form I to Form II in the obtained product is about 50:50.

By suitably adjusting the temperature, distillation time and vacuum conditions, the desired ratio of Form I and Form II can be obtained in the mixture.

Other ratios of the crystalline Forms I and II ranging from about 15% to 85% w/w of Form I in Form II, or Form II in Form I, can be prepared by varying the temperatures, concentration and the duration accordingly.

### Step (C)—Isolating the Desired Mixture of Forms.

The concentrated solution obtained in step (b) may be further maintained at temperatures lower than the concentration temperatures such as for example below about 10° C. to about 25° C., for a period of time as required for isolation of the product. The exact cooling temperature and time required for complete isolation can be readily determined by a person skilled in the art and will also depend on parameters such as concentration and temperature of the solution or slurry.

In an embodiment of the present invention, the cooling of the concentrated solution obtained in step (b) for isolation is carried out slowly in small decrements. Suitably, the temperature is brought down at a rate of 10° C. per hour, or about 5° C. per hour for better results. Slow cooling results in formation of an intimate and uniform mixture of forms. Rapid cooling of the reaction concentrated solution from step (b) may result in disturbance of the ratio of forms.

The mixture of forms obtained from step (b) can be collected from the equipment using techniques such as filtration by gravity, or by suction, centrifugation, and the like or by scraping, or by shaking the container when the solvent is fully concentrated and the material is not filterable; or using a technique specific to the particular apparatus employed.

The crystals so isolated will carry a small proportion of occluded mother liquor containing a higher percentage of impurities. If desired the crystals can be washed on the filter with a solvent to wash out the mother liquor.

The wet cake obtained in step (b) may optionally be further dried. Drying can be carried out with or without applying vacuum at temperatures such as about 35° C. to about 70° C. Drying can be carried out for any desired time period that achieves the desired product purity, such as times about 1 to 20 hours, or longer. Drying may also be carried out for shorter or longer periods of time depending on the product specifications. Drying can be suitably carried out in a tray dryer, vacuum oven, air oven, or using a fluidized bed drier, spin flash dryer, flash dryer and the like. The dried product can optionally be milled to get the required particle size.

Milling or micronization can be performed prior to drying, or after the completion of drying of the product. The milling operation reduces the size of particles and increases surface area of particles by colliding particles with each other at high velocities.

Drying is more efficient when the particle size of the material is smaller and the surface area is higher, hence milling can be performed prior to the drying operation. Milling can be done suitably using jet milling equipment like an air jet mill, or using other conventional milling equipment

An embodiment of the present invention provides intimate mixtures of desloratadine polymorphic Forms I and II that are stable and well suited for use in pharmaceutical formulations.

The term “stable” is used to describe an intimate mixture of desloratadine Form I and Form II that maintains the initial weight ratio of forms during formulation into a pharmaceutical dosage form and thereafter during a commercially useful period of normal storage and use, such as about one year, about 18 months, about two years, or any other desired period. Stability is typically indicated by maintenance of the weight ratio of forms during stability testing, involving storage under standard conditions, such as those described in Test 1150 “Pharmaceutical Stability,” United States Pharmacopeia 29, United States Pharmacopeial Convention, Inc., Rockville, Md., 2005, at pages 2994-2995. Stability testing procedures are well known in the pharmaceutical industry.

In an embodiment of the present invention, the intimate mixtures of desloratadine polymorphs Form I and Form II prepared according to the process of the present invention have low amounts of residual organic solvent. In an embodiment of the present invention, the intimate mixtures of desloratadine polymorphs Form I and Form II prepared according to the process of the present invention contains less than about 3500 ppm of cyclohexane. In an embodiment of the present invention, the intimate mixtures of desloratadine polymorphs Form I and Form II prepared according to the process of the present invention contains less than about 3000 ppm of cyclohexane. In an embodiment of the present invention, the intimate mixtures of desloratadine polymorphs Form I and Form II prepared according to the process of the present invention contains less than about 1000 ppm of cyclohexane. In an embodiment of the present invention, the intimate mixtures of desloratadine polymorphs Form I and Form II prepared according to the process of the present invention contains less than about 200 ppm of individual residual organic solvents. In an embodiment of the present invention, the intimate mixtures of desloratadine polymorphs Form I and Form II prepared according to the process of the present invention contains less than about 100 ppm of individual residual organic solvents.

In an embodiment of the present invention, the intimate mixtures of desloratadine polymorphs Form I and Form II prepared according to the process of the present invention is substantially pure. By “substantially pure” it is meant that desloratadine mixture of forms prepared in accordance with the present invention contains less than about 0.5%, or less than about 0.1% of the corresponding impurities like deschloro desloratadine, bromo desloratadine, dehydro desloratadine, and the starting material as characterized by a high performance liquid chromatography (“HPLC”) chromatogram obtained from a mixture comprising the desired compound and one or more of the said impurities. The percentage here refers to the area-% of the peaks representing the said impurities.

As used herein the term “deschloro desloratadine” refers to 11-(4-piperidinyllidene) 6,11-dihydro-5H-benzo[5,6]cyclohepta[1,2-b]pyridine, represented by Formula Ia;

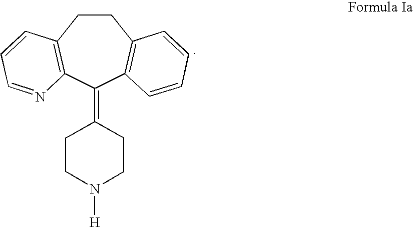

The term “bromo desloratadine” refers to 8-bromo, 11-(4-piperidinyllidene) 6,11-dihydro-5H-benzo[5,6]cyclohepta[1,2-b]pyridine, represented by Formula Ib;

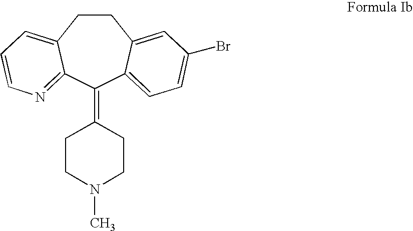

The term “dehydro desloratadine” refers to 8-chloro-11-(4-piperidinyllidene)-benzo-[5,6]cyclohepta[1,2-b]pyridine, represented by Formula Ic

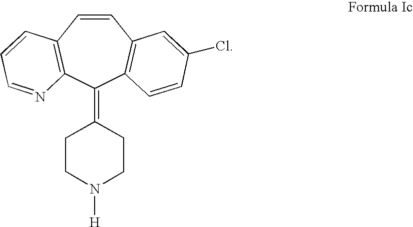

The starting material is chemically known as 8-chloro-11-(1-carboethoxy-4-piperidinylidene)-6-11, dehydro-5H-benzo[5,6]cyclohepta[1,2-b]pyridine is represented by Formula Id.

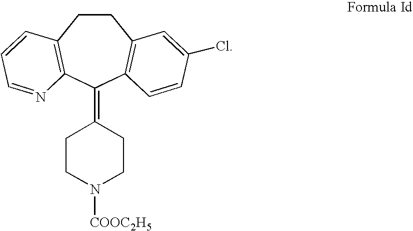

In an embodiment of the present invention, the intimate mixtures of desloratadine polymorphs Form I and Form II typically have mean particle sizes less than about 50 μm or less than about 30 μm. If smaller particles and/or specific particle size distributions are required for subsequent processing, such as formulation into pharmaceutical dosage forms, appropriate size reduction procedures such as milling, grinding, etc. can be used. In addition, classification procedures can be used to separate certain particle size fractions.

The D₁₀, D₅₀ and D₉₀ values are useful ways for indicating a particle size distribution. D₉₀ refers to the value for the particle size for which at least 90 volume percent of the particles have a size smaller than the value. Likewise D₅₀ and D₁₀ refer to the values for the particle size for which 50 volume percent, and 10 volume percent, of the particles have a size smaller than the value. Methods for determining D₁₀, D₅₀ and D₉₀ include laser light scattering, such as using equipment from Malvern Instruments Ltd. (of Malvern, Worcestershire, United Kingdom).

In an embodiment of the present invention desloratadine intimate mixtures of forms have D₁₀ less than about 10 μm. In an embodiment of the present invention desloratadine intimate mixtures of forms have D₁₀ less than about 20 μm. In an embodiment of the present invention desloratadine intimate mixtures of forms have D₅₀ less than about 25 μm. In an embodiment of the present invention desloratadine intimate mixtures of forms have D₅₀ less than about 40 μm. In an embodiment of the present invention desloratadine intimate mixtures of forms have D₉₀ less than about 50 μm. In an embodiment of the present invention desloratadine intimate mixtures of forms have D₉₀ less than about 300 μm. There is no specific lower limit for any of the D values.

An embodiment of the present invention provides intimate mixtures of desloratadine polymorphs Form I and Form II prepared according to the process described in this invention which have a bulk density of less than about 0.3 g/ml. An embodiment of the present invention provides intimate mixtures of desloratadine polymorphs Form I and Form II which have a bulk density of less than about 0.5 g/ml before tapping. An embodiment of the present invention provides intimate mixtures of desloratadine polymorphs Form I and Form II which have a bulk density of less than about 0.5 g/ml after tapping. An embodiment of the present invention provides intimate mixtures of desloratadine polymorphs Form I and Form II which have a bulk density of less than about 1.0 g/ml after tapping. The bulk densities are determined using Test 616 “Bulk Density and Tapped Density,” United States Pharmacopeia 24, pages 1913-4 (United States Pharmacopeial Convention, Inc., Rockville, Md., 1999).

In a still another embodiment, the present invention provides a pharmaceutical composition of an intimate mixture of desloratadine polymorphs Form I and Form II in a weight to weight ratio as described above, prepared by the processes of the present invention and combining the mixture with at least one pharmaceutically acceptable excipient to obtain a pharmaceutical composition.

In an embodiment of the present invention, the intimate mixtures of crystalline Form I and Form II of desloratadine are ideally suited for incorporation into pharmaceutical compositions. According to this embodiment of the present invention there is provided a pharmaceutical composition which contains at least one pharmaceutically acceptable excipient in addition to the desloratadine.

The different pharmaceutically acceptable excipients which can be added to the pharmaceutical composition include but are not limited to diluents, binders, disintegrants, wetting agents, glidants, colorants, emulsifiers, coating agents, thickening agents, antioxidants, preservatives, buffering agents, crystallization preventing agents and the like, depending upon the kind of pharmaceutical composition envisaged.

The mixtures of polymorphic forms of the invention can be incorporated into pharmaceutical compositions such as for example tablets, mini tablets, capsules, powders, granulates, aggregates, suppositories, sachets, troches, lozenges and the like. Controlled release formulations comprising the combinations of the invention are also possible. Liquid formulations in which the combinations are utilized are also envisaged such as for example syrups, suspensions, dry powders for suspension, and the like.

The present invention provides a process suitable for use on an industrial scale for preparation of formulations/compositions of desloratadine. Desloratadine may be crystallized as an intimate mixture of polymorphs in such a way that the ratio between the polymorphs is consistent. As used herein, a “consistent ratio” (or consistent intimate mixture) refers to a ratio of Form I to Form II (wt/wt) that varies within a range of less than about +±15% (wt/wt) between lots, as measured by XRPD.

In an embodiment of the present invention, the pharmaceutical compositions comprising the mixtures of crystalline Form I and Form II of the invention are useful in the treatment of nasal and non-nasal symptoms of seasonal allergic rhinitis. These and other maladies are described in U.S. Pat. No. 4,659,716 and are all included herein by reference.

Certain specific aspects and embodiments of the present invention will be explained in more detail with reference to the following examples, which are provided by way of illustration only and should not be construed as limiting the scope of the invention in any manner.

### EXAMPLE 1

### Preparation of Desloratadine

Toluene (365 liters) was taken into a reactor and 8-chloro-6,11-dihydro-11-(1-methyl-1-piperidylidene)-5H-benzo[5,6]cyclohepta[1,2-b]pyridine (27 kg) was added to it at about 25 to about 35° C. The reaction mass was maintained at about 25 to about 35° C. for about 20 minutes. Triethyl amine (41 liters) was added to the above reaction mass at about 25 to about 35° C., and the reaction mass was heated to a temperature of about 75° C. A solution of ethylchloroformate (79 liters) and toluene (41 liters) was prepared and then added to the above reaction mass at a temperature of about 75° C. The reaction mass was then maintained at about 75° C. for about 90 minutes. Reaction completion was checked using thin layer chromatography. After the reaction was completed, the reaction mass was cooled to a temperature of about 25° C. and water (203 liters) was added followed by addition of caustic lye (11 liters). The reaction mass was stirred at about 25 to about 35° C. about 30 minutes. The pH of the reaction mass was ensured to be above 9.0, and the layers were separated. The aqueous layer was extracted into 135 liters of toluene. The combined organic layer was washed with water (406 liters) in two equal lots. The organic layer was distilled off at about 70° C. under a vacuum of about 600 mm/Hg until about 400 liters of toluene was distilled off. The remaining residue was cooled to about 35° C. and further subjected to distillation in an ATFD at about 65° C. and a vacuum of about 650 mm/Hg to distill off the solvent completely.

The dried powder obtained from ATFD was collected and cyclohexane (250 liters) was added. The mixture was heated to about 75° C. and checked for clear dissolution. After clear dissolution was obtained, the solution was cooled to about 45° C. and maintained for about 25 minutes. Then the reaction mass was then cooled to about 35° C. and maintained for about 30 minutes. The reaction mass was then further cooled to about 12° C. and maintained for about 30 minutes, and then filtered. The filtered solid was washed with cyclohexane (20 liters). The wet solid was dried at about 65° C. under a vacuum of about 600 mm/Hg for about 3 hours. The dry material was then milled in a multi mill to yield 25 kg of the title compound.

Purity by HPLC: 99.9%.

### EXAMPLE 2

### Process for the Preparation of Desloratadine Using Water as Solvent

Sodium hydroxide (51 g), water (200 ml), tertiary-butyl ammonium bromide (12.5 g) and 4-(8-chloro-5 6-dihydro-11 h-benzo-(56)-cyclohepta-(1,2b)-pyridin-11-ylidene-1-piperidiniecarboxylic acid ethyl ester (50 g) were taken into a clean and dry autoclave vessel followed by heating to about 130 to about 140° C. The resultant reaction mixture was agitated at about 3 Kg/cm² of atmospheric pressure for about 5 to about 6 hours. After completion of the reaction, the reaction mass was cooled to about 65° C., and water (400 ml) was added. The reaction mass was stirred for about 30 minutes followed by extraction with toluene (2×100 ml) and the combined organic phase was washed with water (200 ml). The obtained neat organic phase was distilled to about 70% to about 75% of its original volume (260 ml) and the resultant suspension was cooled to about 0 to about 5° C. followed by stirring for about 45 minutes. The separated solid was filtered and the solid was washed with toluene to afford the title compound.

### EXAMPLE 3

### Preparation of 75:25 Ratio of a Combination of Desloratadine Polymorphic Forms I and II

Water (10 liters) and methanol (90 liters) were taken into a reactor and stirred for about 10 minutes. Sodium hydroxide flakes (26.2 Kg) were added to the above mixture below 50° C. The reaction mass was stirred for about 15 to about 20 minutes at a temperature of below 50° C. 25 Kg of 4-(8-chloro-5 6-dihydro-11H-benzo-(56)-cyclohepta-(1,2b)-pyridin-11-ylidene-1-piperidiniecarboxylic acid ethyl ester (loratadine) was added to the above reaction mass followed by heating to about 85° C. The reaction mass was maintained at a temperature of about 85° C. for about 5 hours. After completion of the reaction, the reaction mixture was cooled to about 65° C. and water (200 liters) was added. The reaction mass was maintained at about 65° C. for about 20 minutes, and then toluene (100 liters) was added. The reaction mixture maintained at about 65° C. for about 20 minutes, and then the organic layer was separated. The aqueous layer was again extracted into toluene (100 liters) at about 65° C. in two equal lots. The combined organic layer was washed with water (300 liters) in three equal lots at a temperature of about 65° C. The organic layer was then distilled off at a temperature of about 75° C. under a vacuum of about 600 mm/Hg to distill off about 140 liters of the solvent. The remaining residue was then cooled to about 2° C. and maintained for about 5 hours. The separated solid was then filtered and washed with chilled toluene (15 liters). The wet material was dried in a cone drier at a temperature of about 65° C. under a vacuum of about 600 mm/Hg for about 6 hours.

The dry solid was taken into a reactor containing cyclohexane (380 liters). The resultant mixture was heated to about 80° C. followed and maintained for about 30 minutes to get clear dissolution. The solution was then filtered and the filtered bed was washed with cyclohexane (70 liters). Cyclohexane (about 300 liters) was distilled off from the solution at about 65° C. without applying vacuum. The remaining residue was cooled gradually at the rate of 10° C. per hour to about 10° C. The reaction mass was maintained at a temperature of about 10° C. for about 3 hours. The separated solid was filtered and washed with chilled cyclohexane (26 liters). The wet material was dried at about 65° C. for about 5 hours under a vacuum of 600 mm/Hg. The dry material was sifted in a 30 No. mesh and then micronized under a pressure of about 6.5 kg/cm², and again dried at about 65° C. and a vacuum of 600 mm/Hg for about 6 hours to yield 12.2 kg of the title compound.

### % of Form II by XRD: 22.2.

### Purity by HPLC: 99.9%.

### Particle Sizes: D₁₀: 2.0 μm, D₅₀: 5.0 μm, D₉₀: 10.0 μm.

Bulk Density Before tapping: 0.27 g/ml, after tapping: 0.52 g/ml.
Residual solvent content: Cyclohexane: 892 ppm, all other solvents: Below LOD.

### EXAMPLE 4

### Determination of Desloratadine Form Ii in Form I by X-Ray Diffraction

Experimental conditions for performing the XRPD analysis for quantification were as follows:

| | |
|---|---|
| Instrument used | Powder X-ray diffractometer |
| Make, Model | Bruker AXS, D8 Advance |
| Goniometer | Theta/Theta vertical |
| Measuring circle | 435 mm |
| Radiation | Cu K α-1 (λ = 1.5406 A°) |
| Tube | 2.2 kW Copper long fine focus |
| Detector | Scintillation counter. |
| Voltage (kV), Current (mA) | 40 kV, 50 mA |
| Scan type | Locked coupled |
| Scan mode | Step scan |
| Divergence slit | 1.0 deg. |
| Antiscattering slit | 1.0 deg. |
| Detector slit | 0.2 mm |
| Synchronuous rotation | On |
| Scan range | 22.64° to 23.44° 2θand |
| | ref peak at 20.70° to 21.62° |
| Step Size | 0.04° |
| Time/Step | 50.0 sec. |
| Calculation: | |

The percentage of Form II in Form I is calculated by the following formula:

% of Form II=[(S2/S1)÷K+(S2/S1)]×100

S1=Area of peak corresponding to Form I

S2=Area of peak corresponding to Form II

K=Response constant for Form II relative to Form I. The areas of the peaks associated with Form I and Form II (characteristic peaks) were measured using Bruker X-ray diffraction evaluation software. From these areas, the response constant for Form II relative to Form I was determined. The value for the K factor was 0.13.

The quantification limit of desloratadine Form II content in Form I is more than 1.0% and less than 85% by weight.

### EXAMPLE 5

### Stability Studies for a Mixture of Desloratadine Form I and Form II in a Ratio of 50:50

A 60:40 weight ratio mixture of desloratadine Form I and Form II prepared using the process of Example 2 was subjected to different conditions to determine the stability of the mixture under stress conditions. The results are tabulated below:

| Storage Condition | Form I to Form II Ratio |
|---|---|
| Long term storage for 24 months. | No change in IR absorption spectra |
| Storage at 25 ± 5° C. and | No change in IR absorption spectra |
| 60 ± 5% RH | |
| Storage at 40 ± 5° C. and | No change in IR absorption spectra |
| 70 ± 5% RH | |

The consistency in the IR pattern after subjecting to the storage condition for the particular time period mentioned in the table shows that there is no interconversion of forms during the storage period.

1. A process for preparing an intimate mixture of desloratadine crystalline Form I and crystalline Form II having a desired ratio, comprising:
a) providing a solution of desloratadine in a hydrocarbon solvent;
b) concentrating the solution obtained in step (a) to a specific volume; and
c) isolating the desired mixture of forms.

2. The process of claim 1, wherein a solvent is cyclohexane.

3. The process of claim 1, wherein concentration in step (b) is carried out by distillation.

4. The process of claim 1, wherein the volume of the solvent is concentrated.

5. The process of claim 1, wherein the concentration of the solution is from about 0.1 g/ml, to about 0.15 g/ml and the obtained mixture of desloratadine crystalline Form I and Form II has a ratio of about 70:30.

6. The process of claim 1, wherein the concentration of the solution is from about 0.05 g/ml to about 0.01 g/ml and the obtained mixture of desloratadine crystalline Form I and Form II has a ratio of about 50:50.

7. The process of claim 1, wherein the concentration of the solution is from about 0.05 g/ml to about 0.1 g/ml and the obtained mixture of desloratadine crystalline Form I to Form II has a ratio of about 60:40.

8. The process of claim 1, wherein the solution obtained in step (b) is maintained until a mixture having a desired ratio of forms is obtained.

9. The process of claim 8, wherein the solution is maintained at temperatures lower than the temperature of concentration.

10. The process of claim 9, wherein the temperature is decreased slowly in small decrements.

11. The process of claim 9, wherein the solution is maintained at temperatures below about 15° C.

12. The process of claim 8, wherein the ratio of Form I to Form II is from about 15:85 to about 85:15 by weight.

13. The process of claim 8, wherein the ratio of Form I to Form II is from about 70:30 to about 30:70 by weight.

14. The process of claim 8, wherein the ratio of Form I to Form II is from about 60:40 to about 40:60 by weight.

15. An intimate mixture of desloratadine crystalline Form I and crystalline Form II having a mean particle size of below about 50 μm.

16. An intimate mixture of desloratadine crystalline Form I and crystalline Form II having a mean particle size of below about 30 μm.

17. An intimate mixture of desloratadine crystalline Form I and crystalline Form II having a bulk density of less than about 0.5 g/ml.

18. A pharmaceutical composition comprising an intimate mixture of desloratadine crystalline Form I and Form II in a desired ratio, and at least one pharmaceutical excipient.

19. The pharmaceutical composition of claim 18, wherein the weight ratio of Form I to Form II of from about 15:85 to about 85:15.

20. The pharmaceutical composition of claim 18, wherein the weight ratio of Form I to Form II is from about 75:25 to about 25:75.

21. The pharmaceutical composition of claim 18, wherein the weight ratio of Form I to Form II is from about 60:40 to about 40:60.
